(19) United States
(12) Patent Application Publication
(10) Pub. No.: US 2010/0125191 A1
(43) Pub. Date: May 20, 2010

(54) MRI AS A THERAPEUTIC DEVICE

(76) Inventor: Nedim Turan Sahin, Boston, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 11/719,484

(22) PCT Filed: Nov. 18, 2005
(86) PCT No.: PCT/US05/41934

§ 371 Date: Aug. 27, 2009

Related U.S. Application Data
(60) Provisional application No. 60/628,928, filed on Nov. 18, 2004.

(51) Int. Cl. A61B 5/055; A61B 6/00

(52) U.S. Cl. 600/411; 600/422; 606/33

(57) ABSTRACT

A method and device comprises a system alike a magnetic resonance imaging system (10) that is used intentionally to deposit energy into a selected volume of interest in a member that can be a body (40) via absorption of electromagnetic radiation such as RF (20) selectively within the volume of interest, and further includes control (11) of the system to operate in two other modes than energy deposition, namely imaging and heat mapping.

### RELATED APPLICATIONS

This application claims the benefit of priority under 35 U.S.C. 119(e) to copending U.S. Provisional Application No. 60/628,928, filed on Nov. 18, 2004, the entire contents of which is incorporated herein by reference.

### FIELD OF THE INVENTION

The present invention relates to non-invasive methods and systems for imparting energy upon an internal part of a substance. More specifically, the invention relates to the controlled use of a side-effect of producing images with Magnetic Resonance Imaging (MRI) machines currently viewed as a limitation, namely the heating of tissue due to RF exposure, in order to impart energy to a specific subset of a substance, for instance to apply therapeutic heating to combat cancerous tumors deep within the brain without dangerous surgery and with minimal effect on surrounding healthy tissue.

The invention further relates to using MRI machines, or machines of a similar nature, to carefully guide and focus heating, by using the machines in at least 3 modes: one to image the item in question, a second to create a heat map of the item, and a third which is the mode mentioned above, namely the heating of some part or all of the substance; thus the machine simultaneously takes on functions of diagnosis, recording, monitoring, and treatment, whereas MRI machines are now only used for diagnosis or in some cases guiding of treatments with other devices.

### BACKGROUND OF THE INVENTION

The present invention has great utility in the treatment of brain tumors through the use of existing MRI machines, although it is also applicable to many other treatments and processes and the use of machines much simpler than MRI machines, as will be discussed later.

The invention improves on the state of therapeutic interventions currently the domain of surgery, toxic drugs, and multi-device highly-skilled interventions, as well as providing a novel use for existing MRI machines by exploiting a property of MRI scanners and turning it into a therapeutic benefit.

### Cancer Treatments—Surgery

Currently, surgery cannot reach remote tissue in many parts of the body. For instance, many brain tumors are deemed inoperable. These tumors often grow and first impair the normal abilities of the patient, and then often kill the patient. Death from brain tumors is very common and very devastating. Primary malignant brain tumors are the second or third most common cause of cancer-related deaths in people aged 5 to 55 in America. Brain tumors can result from many kinds of cancer in many parts of the body, for instance skin cancer (especially melanoma) and breast cancer, since cancer cells swim through the blood and spread (metastasize) to the brain.

The reason many of these tumors cannot be treated by surgery is mostly because of their location. Surgery in these regions would be too damaging or disruptive to the region itself (often causing death, because the region is so important), would be too damaging to regions of the brain that surround the tumor (usually if the tumor is very deep in the brain, so the surgeon cannot get access to it except through other brain regions), or would cause bleeding that could not be stopped and would kill the patient. Inoperable areas include the brain stem, thalamus, motor area, regions near the midline venous sinus, and deep areas of gray matter.

Furthermore, surgery, even when possible, involves many risks and disadvantages as a type of therapy. In all surgery especially brain surgery there are risks of: unexplained death, clot formation, serious incidental infections, malpractice of many sorts, heart attack, paralysis, serious scarring, and many other eventualities. Brain surgery specifically can cause loss of major functions, such as speech, sight, hearing, smell, balance, motor coordination, intelligence, and other mental faculties or senses. Also, the obvious fact that the brain is located within the head means that scarring can be particularly noticeable and disfiguring. Some surgeries require peeling back of some of the facial skin, breaking of bones, and thereby accessing parts of the brain lying behind the face. Many modern surgeries can now be done with a relatively small device (endoscope) inserted into the region, such as the brain, but endoscopic surgery still has some risks of infection and complication, and cannot reach all operable parts of the brain.

Aside from risks and side-effects of surgery, there are other disadvantages. Surgery of brain tumors by nature requires the time of one of the most highly-skilled professionals in existence, the brain surgeon, not to mention the others in the operating room such as the anesthesiologist. This means a great deal of waiting time for the patient in order to get scheduled for surgery, a great deal of expense to the patient or the insurance and healthcare system, and strict selection criteria and triage to determine who receives surgery. The recovery time from major surgery also means expensive hospital stays, disruption of family and work life, and exposure to iatrogenic illnesses in the hospital environment.

### Cancer Treatments—Chemotherapy

While surgery is the first treatment option in the case of many brain tumors, another type of treatment offered on its own or in conjunction with other treatments is chemotherapy. Chemotherapy is a broad term that covers all forms of treatment by administering drugs. However, it is usually meant to encompass repeated doses of specific classes of anti-cancer drugs, which mostly work to kill rapidly-dividing cells through some sort of toxicity. On the whole, chemotherapy drugs are not very specific nor specifically delivered. They are administered to the patient, and cells in the patient's body that are dividing at the time are killed or damaged. This preferentially affects cancer cells, since they divide rapidly, but also affects other rapidly-dividing cells such as those that grow hair and nails and line various parts of the body, and the effects extend to some degree to all parts of the body. Chemotherapy is a fairly simplistic and brutal medical practice. Modern techniques for injection or chemical targeting of chemotherapeutic drugs lessen the broad-based effects, and chemotherapy is not as invasive as surgery, but the process is still toxicity and cell death, and still extends to cells well outside of the cancerous target. Akin to the scarring associated with invasive surgery, chemotherapy has drastic visible side effects. Since cells that produce hair divide rapidly and are thus very affected by the procedure, patients often lose their hair from the therapy. This is not only distressing but it is a psychological reminder to the patient that they have cancer, and a signal to employers, friends, and community members that the patient has what might otherwise be able to be a private disorder, and battle. Effects on other parts of the body lead to profound physical sickness and nausea.

### Cancer Treatments—Radiotherapy and Gamma Knife

Still less invasive techniques exist for combating brain tumors. Several techniques do not require any incisions or cuts in the skin or in the brain, and do not even require the injection or ingestion of drugs as in chemotherapy. A class of such techniques uses ionizing radiation to kill cells in the tumor. Most commonly, x-rays and gamma rays are employed. For instance, a whole body region can be intentionally exposed to high doses of these kinds of radiation. Many cancer cells are particularly susceptible to radiation and will die more readily than other cells, though this technique has many of the disadvantages of generalized chemotherapy, namely non-specificity and damage to other tissues. More controlled radiotherapy includes stereotaxic radiosurgery, where a stereotaxic device is used to precisely guide a radiation emission device, which then gives a high dosage of radiation right into the target. Another device that is similar but has its own applications, surgical subculture, and literature is the Gamma Knife, which is a device for very precisely delivering gamma rays to deep tissues. This kills the cells. A surgeon operates the device, which is very large and very expensive and is not installed at all hospitals.

All radiotherapy devices share the disadvantage that they use ionizing radiation which causes DNA damage in cells, and can lead to cancer. Therefore the treatment both creates and destroys cancer, potentially. Normally this is controlled as best as possible in the procedures, and the dosages are so high in the target regions that the cells are just killed outright rather than allowed to remain in a mutated cancerous or pre-cancerous state, and dosages are kept low in non-target areas. However, there is still danger to the patients from exposure to ionizing radiation. Also these is danger to operators of the machinery, which results both in some negative health effects in the caregivers, and many expenses associated with making the machines acceptably safe, and protecting a section of the hospital where radiosurgery is practiced. The gamma knife and similar devices also require highly-skilled operators, who are essentially performing surgery with radiation-based knives, which again leads to issues of expense and difficult scheduling.

### Cancer Treatments—Imaging-guided Hyperthermic Therapy

Yet another class of techniques exists for battling brain tumors and other disease states, which is even less invasive. It is important point to note here, however, that all of the techniques so far discussed exist simultaneously even in the most modern of medical centers. Each technique has its applicability, they are often used in combinations, and each represents a subfield of medical practice that has undergone much development and evolution over the years, and none is the answer for all patients.

This least-invasive class of techniques for treating brain tumors and other such conditions, can be broadly characterized as imaging-guided therapies. Specifically there are cryosurgery techniques, where target tissue is frozen, and there are several types of hyperthermic therapies, where target tissue is heated. These techniques all generally use one apparatus to image the region and supply spatial coordinates for the target tissue, and another apparatus or set of apparatuses to provide the thermal treatment. The devices for thermal treatment are operated by highly-trained personnel, usually surgeons, although some automated versions in development or early deployment may reduce the required skill level of the operator.

Cryosurgery involves cooling a target tissue, and the cold or frozen tissue can cause damage to surrounding tissue if that tissue also becomes cooled to a critical temperature. U.S. Pat. No. 6,032,068 discloses a use of MRI in cryosurgery of cancer. However, this technique only uses the MRI machine for monitoring the temperature of the ice ball within the tissue, and thus is a monitoring technique, not a therapeutic technique directly. U.S. Pat. No. 5,433,717 recites the use of MRI in tissue temperature measurement, also for cryosurgery, by employing T1 measurements to determine temperature of unfrozen tissue regions, but again just for monitoring and not for direct treatment.

Hyperthermia surgery involves increasing the thermal energy in a sample, such as a tumor. The heat itself can kill or damage the cells. Also, heat can be used to increase the effects of other interventions such as chemotherapy. U.S. Pat. No. 6,418,337 recites a use of MRI in hyperthermia surgery. In this invention, MRI is used to make 3-dimensional images of the tumor, and meanwhile heating is provided by another source, namely a laser beam that is conducted to the region by an optical fiber. The laser coagulates the tissue, and provides the treatment; the MRI device is used simply for supplying target coordinates and guiding the delivery of the laser energy. U.S. Pat. Appl. No. US2002/0193682 A1 recites another use of MRI in hyperthermia surgery. This invention also uses MRI to provide images and guiding, and uses an optical fiber to deliver laser energy to coagulate target tissue. Again, the MRI is used for guidance, not for heating or treatment, and likewise the treatment is provided by a separate device that must be introduced into the MRI room (and made safe to do so), and must be operated by a skilled surgeon or operator.

Aside from laser, another device employed to cause remote heating and destruction of unwanted tissue is ultrasound. Ultrasound is a useful technique for tissue disruption and ablation because it is truly non-invasive: unlike laser it does not have to be delivered directly to the tissue for instance by an optical fiber, and unlike ionizing radiation it does not cause mutative damage to cells along the way to the target. U.S. Pat. Appl. No. US2004/0039280 A1 discloses a use of MRI in a system to perform tissue ablation using ultrasound. Furthermore, this invention adds the functionality referenced above, of measuring the heat of the sample with the MRI signal that is recorded from the sample. Thus this system combines heating as well as monitoring of heating. However, the invention shares several disadvantages with other techniques reviewed here. For instance, the method requires both the MRI device and a separate device for depositing energy into the tissue. The separate device in this case is an ultrasound device. Because the MRI involves a magnetic field so strong it can make deadly projectiles out of metal objects, let alone disrupt many types of electronic circuitry, it requires significant engineering to manufacture any medical device to introduce into the MRI system. Furthermore, the ultrasound device needs to be operated by a surgeon or other highly skilled operator. Also, in the preferred embodiment the MRI system is an open-magnet system, allowing the ultrasound operator to access the patient, yet these systems are rare and expensive. Also, ultrasound can be a fairly low-resolution technique that can easily cause bleed-over of energy into surrounding tissues. Finally, while various methods that currently exist attempt to yoke the ultrasound device to the information gained from the MRI scanning, ultimately this is only in terms of providing coordinates to inform the targeting of the ultrasound, and then the heat actually deposited into the sample. For instance, the system does not automatically guide and focus the deposition of the heating into the tissue.

### MRI—Installed Base

MRI machines are very expensive, and installed in most major hospitals in the US, and many through out the world. The purchase, installation, and maintenance of MRI machines is a major cost and technical hurdle for health care centers, but it is one that has been largely born already. The installed base of MM scanners is on the order of thousands or tens of thousands of units, spread about the country, and indeed around the world. Furthermore, there is an installed base of technicians, radiological assistant, and radiologists who are intimately familiar with how to run the scanners.

### MRI—Utilization and Reimbursement

Currently MRI is used only as a diagnostic or monitoring device. As such there is a specific reimbursement and payment structure for time on the machines, according to hospital administration practices, and conventions of insurers. If the machines served a therapeutic purpose, indeed a surgical one, then the reimbursement structure might change. For this reason, and simply because more procedures could be carried out on these expensive machines, hospitals may get a better return on their investment in the machines.

Furthermore, the fact that MRI has not yet been used as a direct energy-deposition device is evidence that the current state of the art teaches away from the current invention. MRI is used predominantly for imaging; those doing thermal surgery treatments use the technology only for the guidance and monitoring of the procedures; and indeed the FDA and MRI device manufacturers take great pains to assure that energy deposition due to the technology of MRI is kept to a minimum. Radio-frequency energy absorption is viewed as a necessary evil of imaging, to force a signal that can be read out from the sample, and currently the technology focuses on the read-out and processing of the signal; while the present invention involves no read-out at all (in the heating mode) and no image-creation, but rather focuses on the excitation phase and uses it to intentionally impart energy precisely within a tissue.

### Remote Deposition of Energy

In a more general sense, the remote deposition of energy into a substance, especially coupled with precise imaging and monitoring of the substance and its surrounds, is very important for many medical and non-medical application, and poses a significant set of technical challenges. At the most basic level, a technique that can deposit energy into a substance generally can be expected to deposit that energy into all parts of the substance, not just the target contained within. Complex strategies can be employed, for instance shining laser or ultrasound from multiple directions so that they converge only in the target tissue, but in any case some energy will be absorbed by tissue on the pathway to the target. This is because the interaction that leads to energy deposition is based on intrinsic properties of the radiant energy and the substance.

In MRI, however, the situation is different. There is a third item involved, namely the magnetic field. It is the combination of the magnetic field and the substance and the radiant energy (radio-frequency electromagnetic radiation (RF) in this case) that must converge in order to cause deposition of energy.

MRI works on the principle of nuclear magnetic resonance (NMR). Within a strong magnetic field, chemicals can experience a special state of resonance. Specifically, the atomic nuclei in the atoms forming the molecules of the chemicals in question have a particular property called their gyromagnetic ratio. This physical property determines the precise frequency at which the nuclei will spin, or “precess,” in a given strong magnetic field. For instance hydrogen atoms in water precess at a frequency of about 63.9 MHz in a magnetic field strength of 1.5 Tesla. This means that such atoms in a field of 1.5 T can absorb energy from RF waves tuned to 63.9 MHz, and they cannot absorb energy from electromagnetic radiation at other frequencies. Once an atom absorbs energy into the nucleus from electromagnetic radiation, it changes what is called its spin state, and then when the energy-supplying radiation is removed, the spin state reverses and some energy is emitted from the nucleus in the form of re-emitted electromagnetic radiation (e.g. radio waves). This echo of the signal is what is read by the MRI machine to get information needed to form an image.

Thus, the magnetic field is required in order to form the image, and in fact it is required for the nuclei to absorb energy from the electromagnetic radiation (EMR). Without the magnetic field, the EMR (especially in the RF range) will mostly pass through samples such as human tissue with minimal interaction. One way of viewing this is that MRI is like taking a picture, but instead of light the system uses RF radiation, and the samples are usually transparent to RF so it is impossible to take pictures of them. However, in the right conditions just the part that is in a certain magnetic field suddenly becomes reflective and then one can take the picture, of just that section. If it is a slice right down the middle of the brain, for instance, that is in the right strength magnetic field for the RF being emitted, then only that slice will absorb and re-emit energy and only that slice will show up in the picture. This is a core principle of MRI. This also means that if the magnetic field is tailored in just such a way to make a tumor experience a magnetic field of a predetermined strength (all at once, or in little pieces of the tumor, in sequence) then it is possible to cause absorption of RF energy only in that tissue, simply in the normal running of the MRI machine. This lies at the core of the present invention, which purports to use the above principle to focus RF or other similar energy intensely on a tumor or on some other tissue or substance wherein heating is somehow useful, and to do so concertedly.

The current prior art in the field of MRI teaches away from using the machines in this way, instead providing ways to avoid or limit RF heating of tissue. The current prior art for remote heating of tissue includes no technique that can so selectively heat just a target, let alone one that can be so neatly coupled with an imaging and monitoring technology.

### DISADVANTAGES OF PRIOR ART

The current methods for treatment of malignant tumors, for remote deposition of energy into a part of a sample, and utilization of MRI machines, all have very many extremely beneficial uses, and yet also suffer from a number of disadvantages.
(a) Surgical treatments involve serious health risks, including death, infection, heart attack, stroke, paralysis, loss of mental faculties.
(b) Surgical intervention may result in disfiguring scarring.
(c) Surgical interventions cannot reach many brain tumors at all.
(d) Surgical treatments often require long and expensive hospital stays, and disruption of work and family life.
(e) Most current interventions require the time and coordination of schedule of highly-skilled medical practitioners such as neurosurgeons, leading to great expense, long wait times, and restricted access to care.
(f) Chemotherapy is slow, does not always work, causes damage to tissue all through the body, and results in hair loss and other visible and uncomfortable symptoms.
(g) Radiation therapy and radiosurgery involve damaging ionizing radiation, lead to health risks from radiation exposure in patients and hospital staff, lead to high costs of treatment due to safety procedures, must be operated by highly-skilled personnel, and can lead to physical sickness and discomfort.
(h) Imaging-guided thermo-therapy techniques provide the least invasive therapy, yet they suffer from key disadvantages in that they require multiple devices to carry out the treatment, they require a skilled operator (within the high magnetic field of the MRI room) to operate the thermal device, they largely do not couple the heating directly to imaging and monitoring in the way possible with an MRI-based total solution, they can be invasive in the case of fiber-optic lasers, they can cause spill-over to surrounding tissue or substance, and any extra device in the MRI room needs to be magnet-compatible.
(i) MRI is currently not utilized as a direct means of depositing energy, nor is it directly used for treatment and therapy, and hospitals have made a huge investment in this device without getting any direct treatment value out of them.
(j) Most current means for depositing heat or other energy into a sample unavoidably affect the areas surrounding the target.
(k) Most current interventions require the time and coordination of schedule of highly-skilled medical practitioners such as neurosurgeons, leading to great expense, long wait times, and restricted access to care.

### SUMMARY OF THE INVENTION

In accordance with the present invention, a method and device comprises a system alike a magnetic resonance imaging system that is used intentionally to deposit energy into a selected volume of interest in a member that can be a body via absorption of electromagnetic radiation such as RF selectively within the volume of interest, and further includes control of the system to operate in two other modes than energy deposition, namely imaging and heat mapping.

The present invention has one or more of the following advantages:
(a) to provide a way to heat remote tissue that does not incur risk of death or major infection for the patient;
(b) to provide a way to heat remote tissue that minimizes risk of stroke, heart attack, paralysis, or loss of mental faculties;
(c) to provide a way to heat remote tissue that does not cause scarring or disfigurement, in the skin or tissue surrounding the target;
(d) to provide a way to heat remote tissue that can reach every type of brain tumor;
(e) to provide a way to heat remote tissue that can reach every tissue in the body, no matter how deep within the body;
(f) to provide a way to heat remote tissue that does not require long hospital stays, and minimizes disruption of work and family life;
(g) to provide a way to heat remote tissue that does not require long wait times before a patient can be scheduled for the procedure, and that can be open to most patients;
(h) to provide a way to heat remote tissue that requires little or no time from a surgeon, and no time from other operating room staff such as anesthesiologists;
(i) to provide a way to heat remote tissue that can be carried out by an operator of medium skill level (for the healthcare field); to provide a way to heat remote tissue that does not result in hair loss, unless that is the desired effect of the treatment;
(k) to provide a way to heat remote tissue that is selectively focused on just the target tissue or tissues;
(l) to provide a way to heat remote tissue that does not involve ionizing radiation;
(m) to provide a way to heat remote tissue that does not require additional major expenditures on the part of the hospital;
(n) to provide a way to heat remote tissue that can be delivered economically to patients in under-funded health care centers, so long as they have an MRI machine;
(o) to provide a way to heat remote tissue that is non-invasive;
(p) to provide a way to heat remote tissue that requires only one primary device;
(q) to provide a way to heat remote tissue that can be accomplished with a machine that can also simultaneously take high-quality images of the tissue and guide and verify the process;
(r) to provide a way to heat remote tissue that can be accomplished with a machine that can also simultaneously measure the heat values of the tissue in all parts and guide and verify the heating process;
(s) to provide a way to use MRI and similar machines to perform actual treatment and therapy, not just guide it or diagnose its need;
(t) to provide a way to heat remote tissue that minimizes or limits heating to surrounding tissue;
(u) to provide a method for non-invasive therapy that does not require the operator to stand within a high-field magnetic device;
(v) to provide a way to heat remote tissue that is fairly quick;
(w) to provide a method for non-invasive therapy or substance heating and monitoring that does not require an operator who is a surgeon or someone equivalently specialized in training; and
(x) to provide a means for heating a substance that is not biological.

Further objects and advantages are to provide a way to activate a heat-activated compound, to power a remote device such as an implantable medical device that can derive power from electromagnetic radiation, to speed healing of bones, to provide heating to a complex tissue conformation such as the skin layers of the face, to cause selective disruption of fat tissue, and to provide a tool for research and innovative medical procedures. Still further objects and advantages will become apparent from a consideration of the ensuing description and drawings.

### BRIEF DESCRIPTION OF THE DRAWINGS

FIG. 1 is a schematic illustration of a system for depositing energy from electromagnetic radiation within a target substance that is an internal part of a member, according to one embodiment of the present invention.

FIG. 2A is a detail from FIG. 1 showing a human head as the member, a tumor as the target substance, a volume of interest surrounding the tumor, and an antenna as the broadcast means.

FIG. 2B is a detail from FIG. 2, showing the volume of interest decomposed into individually addressable volume elements, or voxels.

FIG. 3A is a high-level flow chart of one process for using this embodiment of the invention.

FIG. 4A schematically shows a means to focus the heating within the target, specifically using a specially inserted member to absorb the electromagnetic radiation within the volume of interest.

FIG. 4B schematically shows a means to focus the heating within the target, specifically using a chemical or property naturally part of or specifically delivered into the volume of interest, and which absorbs the electromagnetic radiation.

FIG. 5A-5K teach and depict another class of means to focus the heating within the target, holding in common that they use only the magnetic fields and electromagnetic radiation emissions associated with traditional magnetic resonance imaging (MRI), with variants of pulse sequences causing spatially selective excitation and thus heating. There is no frequency-encoding gradient, and no phase-encoding gradient. There is no read-out period. The signal is ignored and not shown in this pulse-sequence diagram because it is not recorded. It may be a series of dampened echos, but that depends a lot on the amplitude and spacing of the RF pulses.

Note that gradient pulses are shown with dotted lines to suggest that they may be of different amplitudes. This is to accommodate the idea that each slice orientation (A, B, C . . . ) may be at a different orientation. The amplitudes are to be interpreted as the absolute-value of the actual gradient prescription, to avoid my having to depict lines going below zero. Also the dashing of the lines begins at an arbitrary point, but any amplitude should be possible, including zero.

FIG. 5A schematically illustrates the magnetic means and electromagnetic radiation broadcast means from FIG. 1, demonstrating the homogenous B(0) fixed magnetic field.

FIG. 5B schematically illustrates one of the gradient magnetic fields, specially the Z gradient, which shares the axis of the fixed magnetic field.

FIG. 5C combines the elements of 5A and 5B, demonstrating an altered net magnetic field with one gradient magnetic field switched on, and noting illustrative points A and B along the net field axis.

FIG. 5D adds a member and demonstrates that slabs of the member would be excited by electromagnetic radiation emitted at frequencies where some compound (such as water) would resonate at the magnetic field strength at the points A or B.

FIG. 5E focuses on a head member and schematically illustrates the full complement of 3 orthogonal gradient magnetic fields.

FIG. 5F illustrates absorption of energy from electromagnetic radiation, accomplished by traditional volume spatially selective excitation pulse sequences.

FIG. 5G is a flow chart for steps involved in a novel pulse sequence that is volume spatially selective.

FIG. 5H is a graphical representation of one general embodiment of the pulse sequence of FIG. 5G.

FIG. 5I is a graphical representation of another general embodiment of the pulse sequence of FIG. 5G.

FIG. 5J is a graphical representation of a more specific embodiment of the pulse sequence in FIG. 5H.

FIG. 5K is a visual schematic of some of the excitation slabs or absorption zones created by one embodiment of the spatially selective pulse sequence outlined in FIG. 5G.

FIG. 6A represents one of many alternate embodiments of the method in FIG. 1, for assisting in the healing of bones.

FIG. 6B illustrates another of many alternate embodiments of the method in FIG. 1, where an implanted device is powered or activated by energy remotely broadcast according to the present invention.

FIG. 7 is a schematic illustration of an apparatus that is a further embodiment of the present invention, and adds to the apparatus of FIG. 1 the ability to take images of and map the temperature of the member.

FIG. 8A is a high-level process chart showing one way of operating the apparatus in FIG. 7, with its primary modes of imaging, heat-mapping, and heating.

FIG. 8B is a visual schematic of a timeline showing operation of the apparatus in FIG. 7, alternating among its primary operational modes according to one embodiment of this process.

FIG. 8C is a detailed version of the process chart in FIG. 8A.

FIG. 9 shows a schematic screen-shot of a graphical user interface included in a control system to operate the apparatus in FIG. 7, according to one embodiment of the invention.

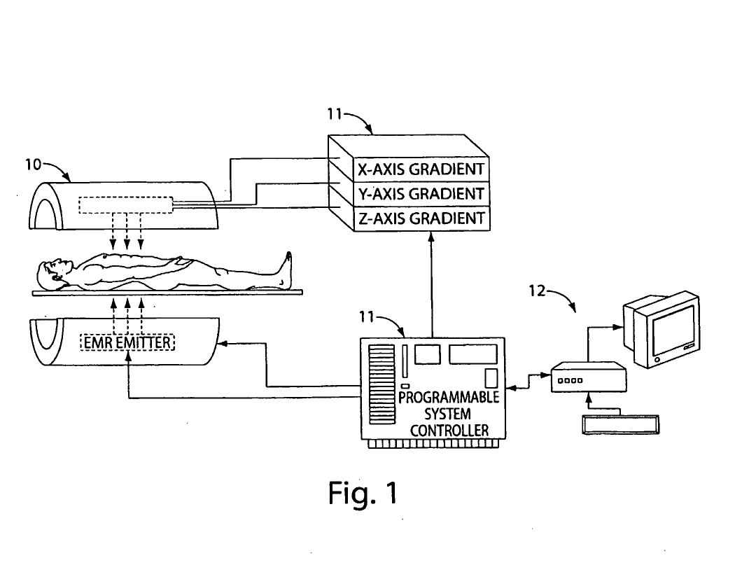

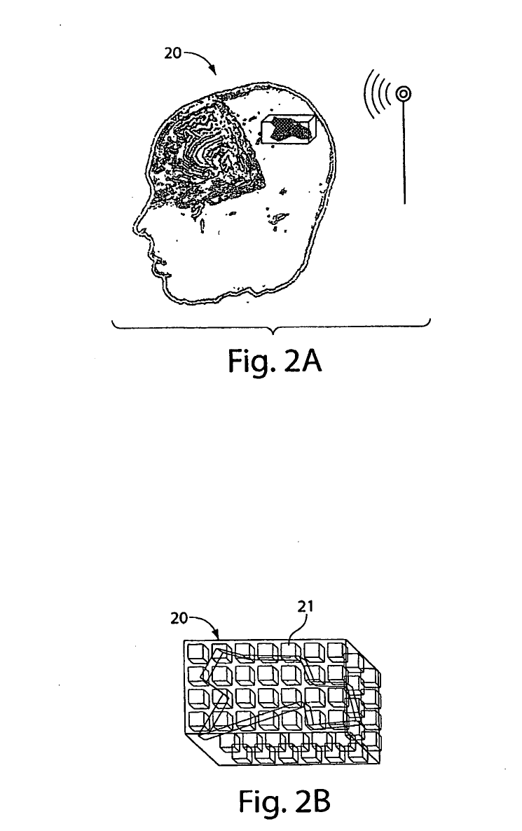

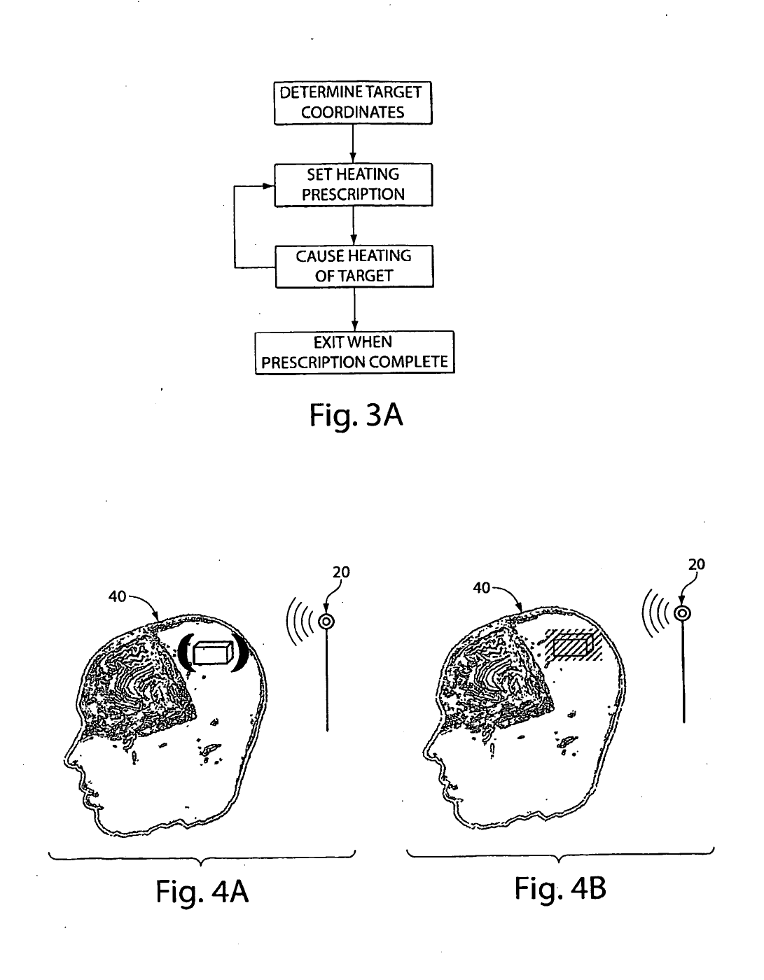

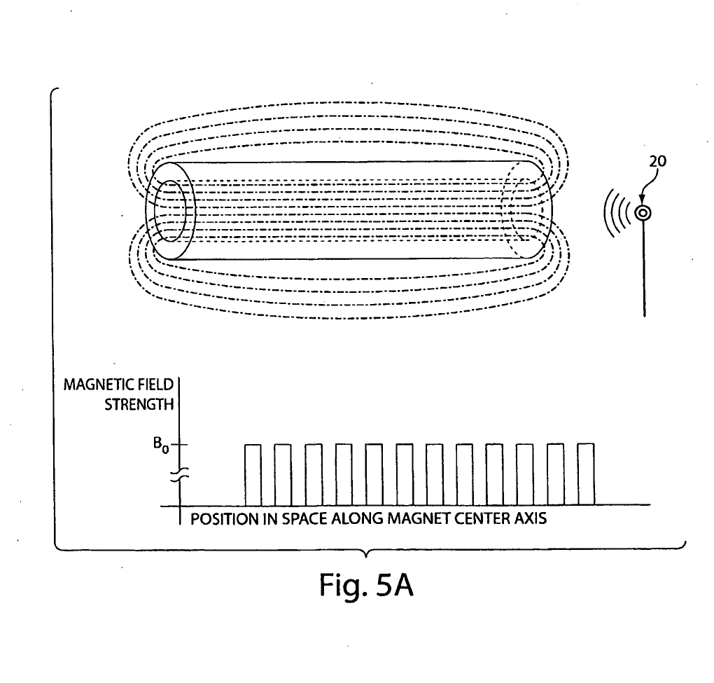

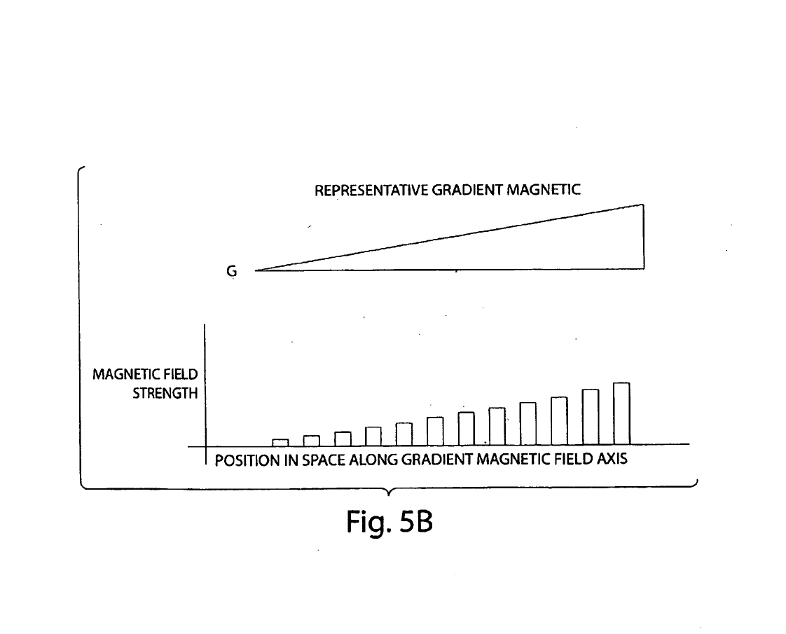

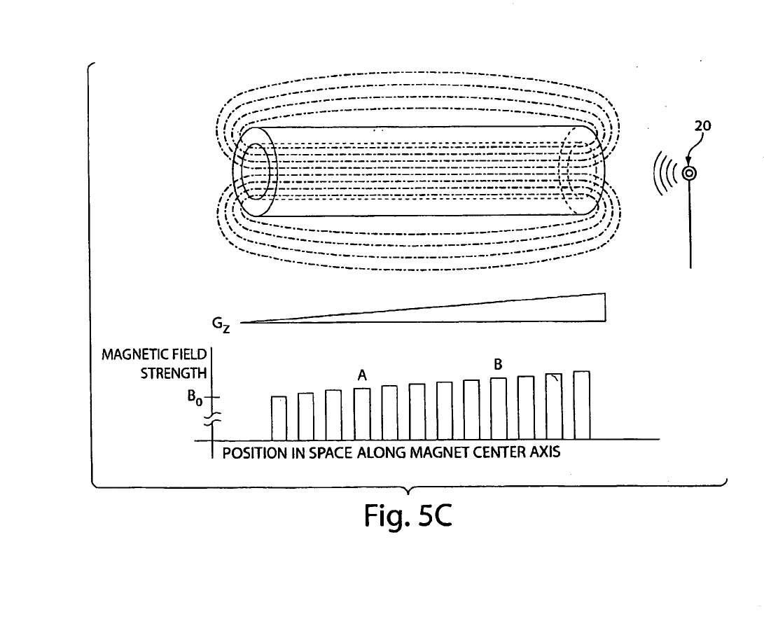

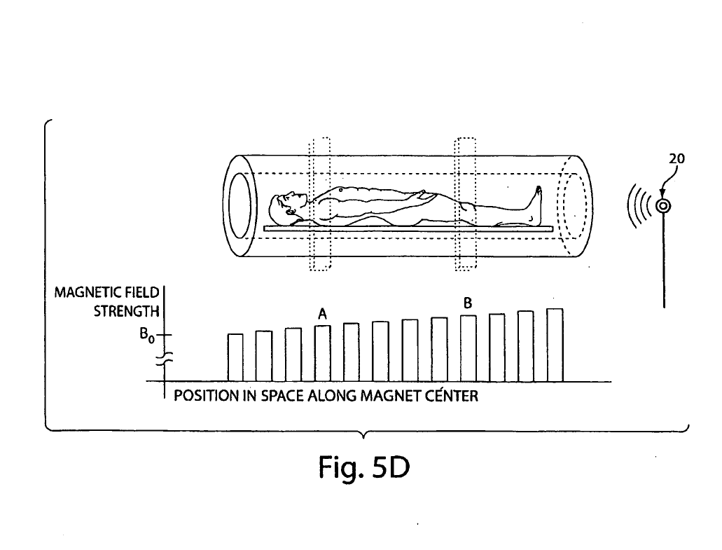

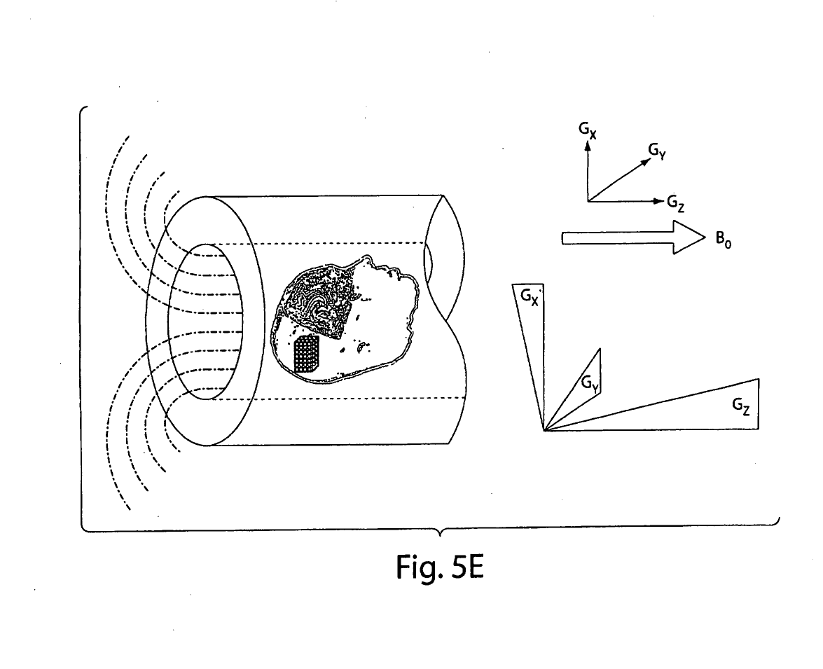

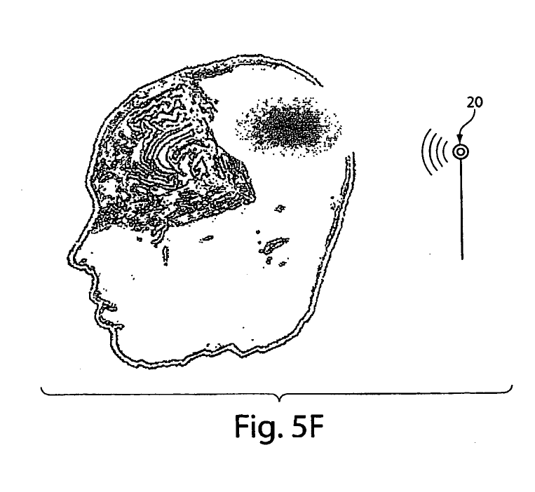

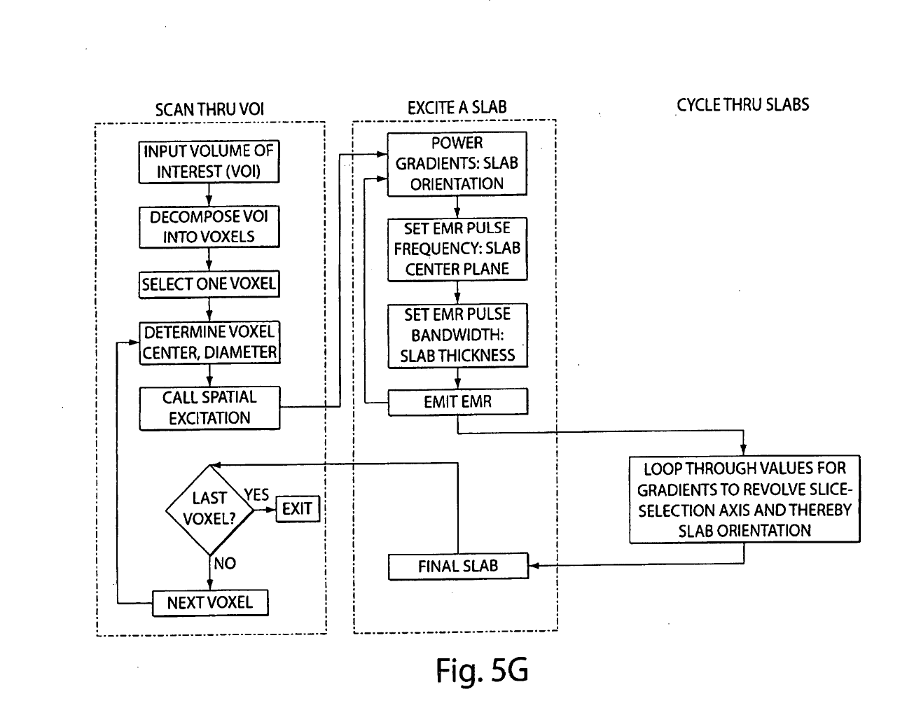

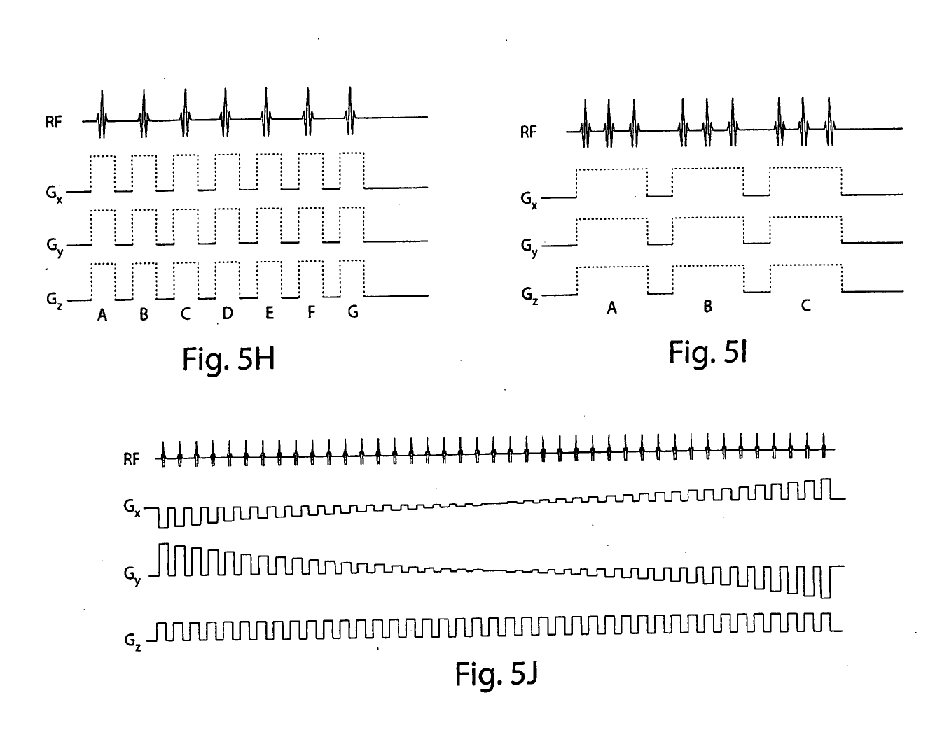

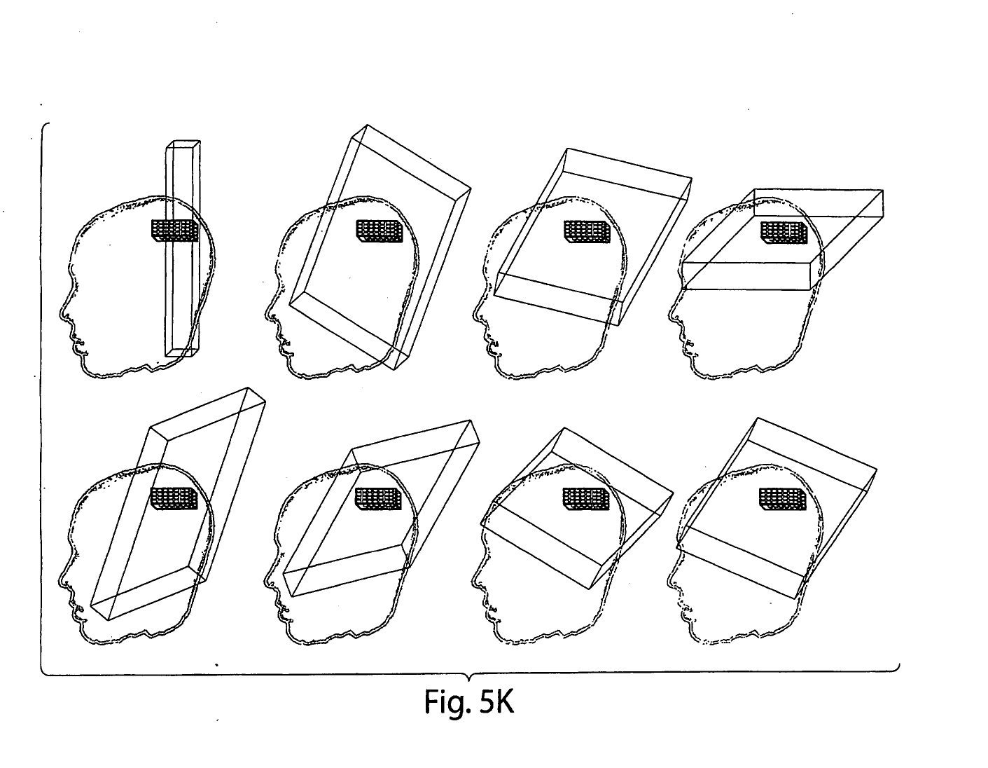

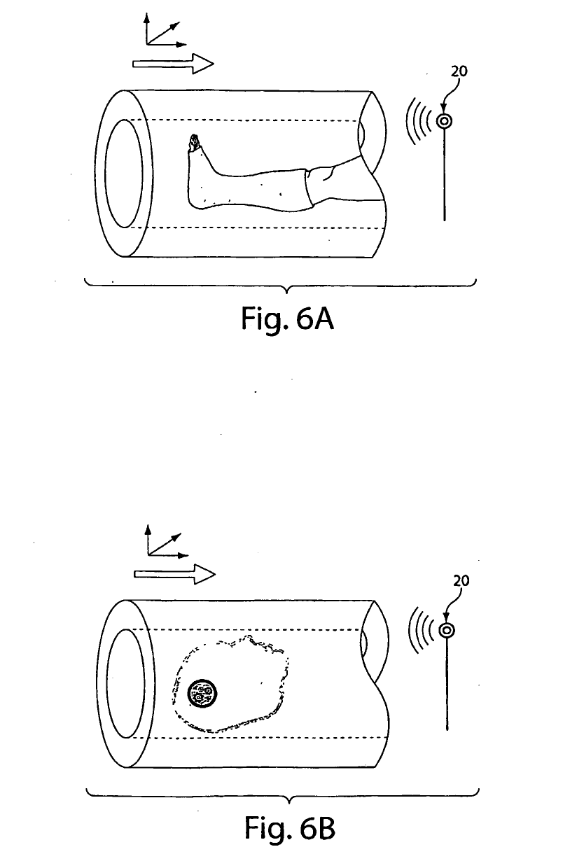

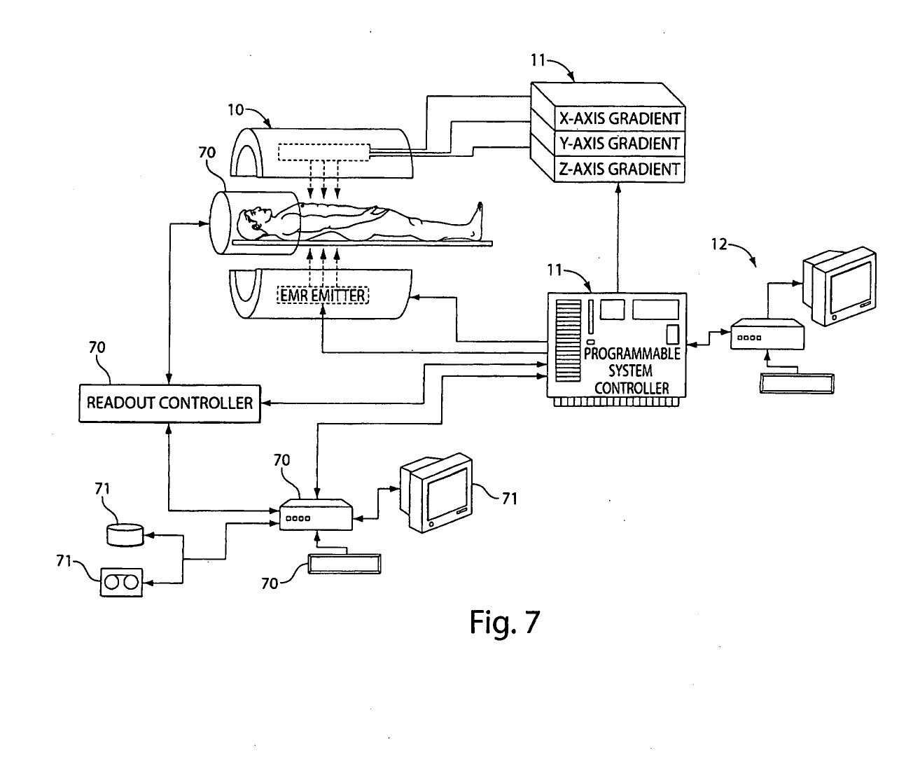

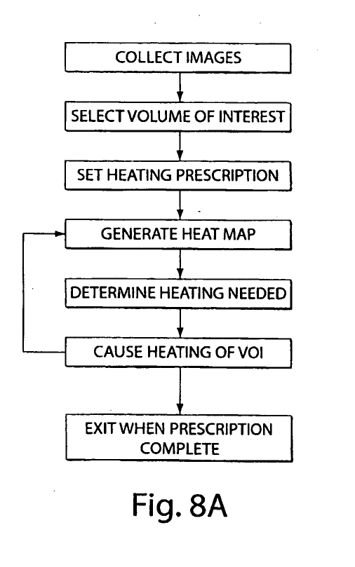

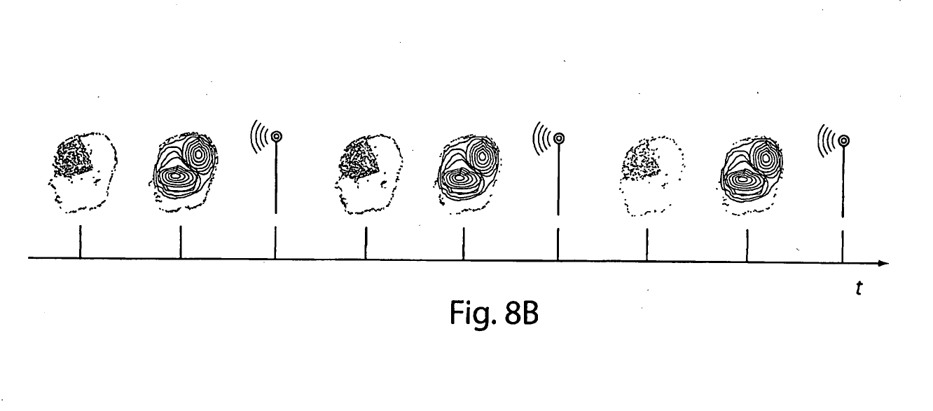

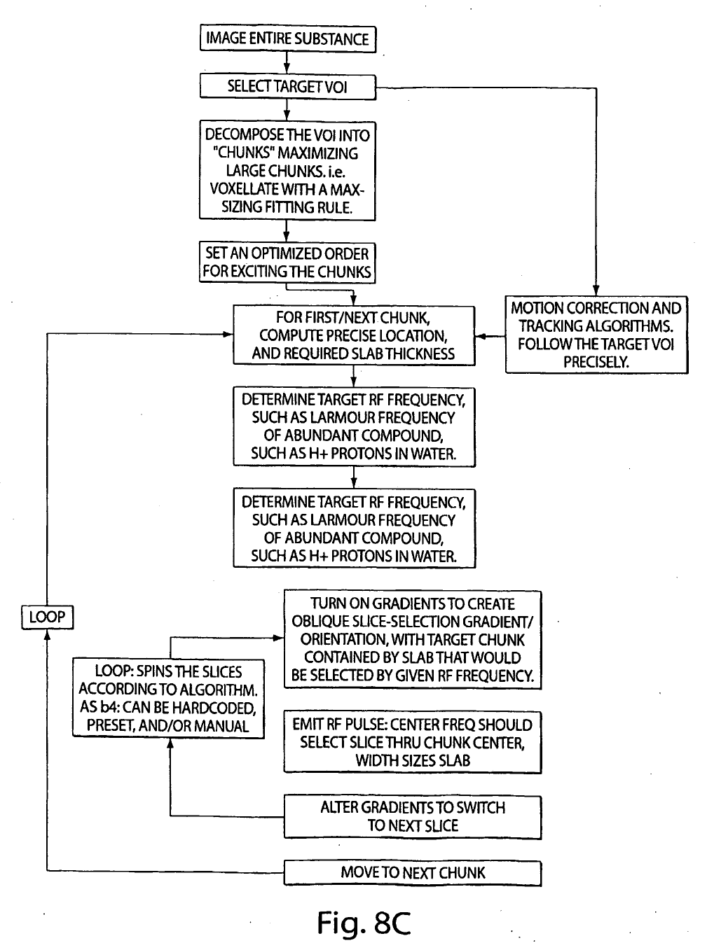

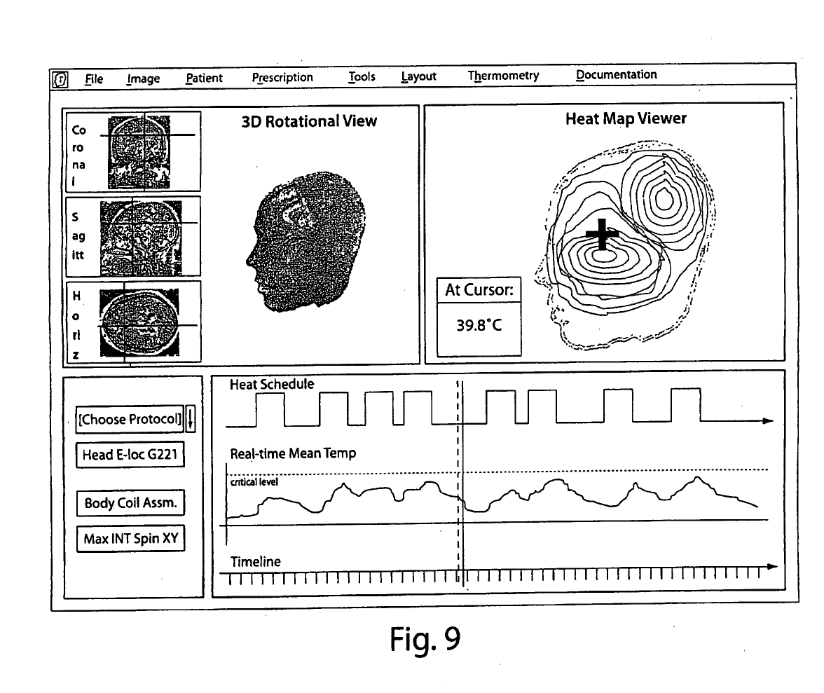

### DETAILED DESCRIPTION OF THE INVENTION

The features and other details of the invention are set forth herein, and described in the appended figures. It will be understood that particular embodiments described herein are shown by way of illustration and not as limitations of the invention. The principal features of this invention can be employed in various embodiments without departing from the scope of the invention.

### KEY TO REFERENCE NUMBERS

102 member
104 receptacle for member
106 electromagnet
108 antenna
112 X-axis gradient system
114 Y-axis gradient system
116 Z-axis gradient system
118 system controller
120 control console computer
122 control console monitor
124 control console input device
200 head member
202 volume of interest
204 target substance
206 antenna
208 emitted electromagnetic radiation
210 single voxel
402 head member
404 volume of interest
406 absorptive insert
408 absorptive compound
702 MR signal receive coil system
704 MR signal readout control system
706 MR signal and image processor
708 image console
710 image console monitor
712 data storage device
714 data archive device

The present invention for depositing energy remotely and non-invasively in a target substance contained within a member, and imaging and heat-mapping the member, has the following advantages:
(a) it provides a way to heat remote tissue that does not incur risk of death or major infection for the patient;
(b) it provides a way to heat remote tissue that minimizes risk of stroke, heart attack, paralysis, or loss of mental faculties;
(c) it provides a way to heat remote tissue that does not cause scarring or disfigurement, in the skin or tissue surrounding the target;
(d) it provides a way to heat remote tissue that can reach every type of brain tumor;
(e) it provides a way to heat remote tissue that can reach every tissue in the body, no matter how deep within the body;
(f) it provides a way to heat remote tissue that does not require long hospital stays, and minimizes disruption of work and family life;
(g) it provides a way to heat remote tissue that does not require long wait times before a patient can be scheduled for the procedure, and that can be open to most patients;
(h) it provides a way to heat remote tissue that requires little or no time from a surgeon, and no time from other operating room staff such as anesthesiologists;
(i) it provides a way to heat remote tissue that can be carried out by an operator of medium skill level (for the healthcare field);
(j) it provides a way to heat remote tissue that does not result in hair loss, unless that is the desired effect of the treatment;
(k) it provides a way to heat remote tissue that is selectively focused on just the target tissue or tissues;
(l) it provides a way to heat remote tissue that does not involve ionizing radiation;
(m) it provides a way to heat remote tissue that does not require additional major expenditures on the part of the hospital;
(n) it provides a way to heat remote tissue that can be delivered economically to patients in under-funded health care centers, so long as they have an MRI machine;
(o) it provides a way to heat remote tissue that is non-invasive;
(p) it provides a way to heat remote tissue that requires only one primary device;
(q) it provides a way to heat remote tissue that can be accomplished with a machine that can also simultaneously take high-quality images of the tissue and guide and verify the process;
(r) it provides a way to heat remote tissue that can be accomplished with a machine that can also simultaneously measure the heat values of the tissue in all parts and guide and verify the heating process;
(s) it provides a way to use MRI and similar machines to perform actual treatment and therapy, not just guide it or diagnose its need;
(t) it provides a way to heat remote tissue that minimizes or limits heating to surrounding tissue;
(u) it provides a method for non-invasive therapy that does not require the operator to stand within a high-field magnetic device;
(v) it provides a way to heat remote tissue that is fairly quick;
(w) it provides a method for non-invasive therapy or substance heating and monitoring that does not require an operator who is a surgeon or someone equivalently specialized in training; and
(x) it provides a means for heating a substance that is not biological.

The invention has the additional advantages in that it can provide a way to activate a heat-activated compound, it can power a remote device such as an implantable medical device that can derive power from electromagnetic radiation, it may be able to speed healing of bones, it can provide heating to a complex tissue conformation such as the skin layers of the face, can cause selective disruption of fat tissue, and it provides a tool for research and innovative medical procedures.

This patent specification contains many specificities, but these should not be construed as limiting the scope of the invention, rather as providing illustrations of some of the presently preferred embodiments of this invention. For example, the invention can be embodied in a machine that is much simpler than a traditional MRI machine but which has the basic parts mentioned below in the claims. Furthermore, many diseases may be treated by means of monitored heating, such as arthritis, psychiatric illnesses, obesity (through targeted ablation of adipose cells), heart disease (by targeted ablation of fat surrounding the heart or plaques within arteries), and other conditions.

### EQUIVALENTS

Those skilled in the art will recognize, or be able to ascertain using no more than routine experimentation, numerous equivalents to the specific procedures described herein. Such equivalents are considered to be within the scope of the invention. Various substitutions, alterations, and modifications may be made to the invention without departing from the spirit and scope of the invention. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the invention and embodiments thereof.

1. A method for depositing energy within an internal part of a member comprising:
a) providing an antenna that can emit electromagnetic radiation;
b) providing a magnetic field, the magnetic field being able to induce nuclear magnetic resonance in at least one composition of matter contained within the magnetic field, whereby a plurality of atomic nuclei in the composition of matter will resonate and can absorb energy from electromagnetic radiation of at least one frequency that can be emitted by the antenna;
c) providing a member containing a target substance;
d) arranging the member within operating range of the antenna and the magnetic field;
e) emitting electromagnetic radiation with the antenna, at a frequency or range of frequencies effective to cause absorption of some energy from the electromagnetic radiation within the target substance; and
f) repeating the emission of electromagnetic radiation one or more times, thereby intentionally causing energy deposition within the target substance;
g) whereby the energy from the electromagnetic radiation can be deposited within the target substance, which can be body tissue, and the energy deposition can cause heating, and for example an apparatus can heat and thereby treat or destroy diseased or cancerous tissue deep within a body, without physical surgery or other invasive intervention.

2. The method of claim 1, wherein the antenna comprises a radio-frequency transmission coil system incorporated into a commercially available magnetic resonance imaging system.

3. The method of claim 1, wherein the magnetic field is produced by one or more electromagnets, or one or more permanent magnets, or a combination of both.

4. The method of claim 1, wherein the magnetic field is provided by devices in a commercially available magnetic resonance imaging system, the devices providing a static magnetic field and/or gradient magnetic fields.

5. The method of claim 1, wherein the member or the target substance comprises a body of a human or of a non-human animal, or one or more parts thereof.

6. The method of claim 1, wherein the target substance comprises at least one biological tissue selected from the group consisting of brain tissue, spinal cord tissue, other neural tissue, cerebrospinal fluid, blood, lymphatic tissue, joints, bones, bone growth plates, bone marrow, arthritic tissue, muscle, veins, arteries, capillaries, heart tissue, lungs, ligaments, tendons, cartilage members, classes of cells, biochemicals, bacteria, viruses, parasites, cancerous tissue, arthritic tissue, transplanted tissue, pathological tissue, lacerated tissue, lesioned tissue, necrotic tissue, epileptic tissue, ischemic tissue, dysmorphic tissue, prolapsed tissue, embryos, hair follicles, skin, dermis, connective tissue, fat, scar tissue, tissue with complex topography such as the deep layers of skin of the entire face, solid waste products, liquid waste products, bodily organs, and organ tissue.

7. The method of claim 1, wherein the member or the target substance comprises at least one item selected from the group consisting of biological samples, animals, plants, food substances, substances containing heat shock proteins, tattoos, semiconductor materials, circuit boards, moldable artistic materials, archaeological samples, prosthetics, plastics, construction materials, cosmetics, liquids, gases, and solids.

8-12. (canceled)

13. The method of claim 1, wherein the energy deposition may be concentrated substantially within the target substance using a special substance to guide and focus absorption of electromagnetic radiation, more specifically:
a) providing one or more absorptive means, which may include devices or compositions of matter, the absorptive means being tuned to selectively absorb electromagnetic radiation of a predetermined frequency or range of frequencies that can be provided by the antenna, within or without presence of the magnetic field;
b) locating the absorptive means selectively in the vicinity of the target substance, whereby heat or other energy may be transferred to the target substance from the absorptive means; and
c) controlling the antenna such that it will emit electromagnetic radiation at the predetermined frequency or range of frequencies;
d) whereby the absorptive means located selectively in the vicinity of the target substance may suffice as a means for focusing localized energy deposition in an internal part of a member, according to the method of the invention.

14. The method of claim 13, wherein the absorptive means comprises an item or property already selectively characteristic of the target substance, such as at least one element chosen from the group containing tissue-specific macromolecules, tissue-specific chemicals, substance-specific dyes, deoxyribonucleic acid sequences, ribonucleic acid sequences, classes of cells, and compositions of matter.

15. The method of claim 13, wherein the absorptive means comprises an item selectively
inserted into or imposed upon the target substance, the item comprising at least one element chosen from the group containing chemicals, drugs, dyes, contrast agents, physical inserts, antennas, liquids, solids, and compositions of matter.

16. The method of claim 13, wherein the absorptive means comprises at least one device that requires power to operate, the device being powered by absorbing energy from the electromagnetic radiation at the predetermined frequency or range of frequencies, the device directly using or converting or storing the energy or a combination thereof.

17. The method of claim 1, wherein location of the target substance is predetermined.

18. The method of claim 1, further providing a localizing means, such as a digital stereotaxic device, for determining location or spatial coordinates of the target substance.

19. The method of claim 1, further providing a guiding means for assuring that all parts of the target substance can receive the energy deposition, or providing a programmable control means for automating the energy deposition according to a schedule or prescription such as may be effective for therapy of a medical condition, or providing a combination of both.

20-24. (canceled)

25. The method of claim 1, further comprising a method for dissipating energy contained or deposited in the target substance or surroundings thereof, or both, for instance in order to limit spreading of deposited energy into substances surrounding the target substance, such as but not limited to:
a) cooling surrounding environment of the member;
b) bathing the member in a cool fluid before, during or after energy deposition;
c) in a case where the target substance is a tissue or bodily region in an organism that has a cardiovascular system, having the organism perform acts that increase the rate or throughput of the cardiovascular system, before, during or after energy deposition, such that more blood may pass through tissue around or in the tissue or bodily region, thereby effecting greater cooling;
d) in a case where the target substance is a tissue or bodily region in an organism that has a cardiovascular system, administering a drug to increase blood flow through tissue around or in the tissue or bodily region, thereby effecting greater cooling;
e) using quick and powerful bursts of electromagnetic radiation to cause a large amount of energy deposition per unit time yet for a short time span, thereby minimizing gross heat transfer and damage to surrounding matter while preserving or enhancing the ability to cause thermal damage to the target substance; or
f) using gradual and small bursts of electromagnetic radiation to cause slow and steady heating, thereby giving the member or target substance time to cool itself according to natural processes;
g) or any combination or combinations thereof.

26. The method of claim 1, including an antenna means, a radio-frequency transmission coil assembly, a fixed magnetic field, gradient magnetic fields, a means for selectively causing electromagnetic radiation absorption involving selective imposition of magnetic fields and electromagnetic radiation to converge on the larmour frequency of a target composition of matter such as but not limited to water hydrogen atoms, a means for operating magnetic field gradients and radio-frequency coils of a magnetic resonance machine to cause selective absorption of radio-frequency energy in a target composition of matter, a means for establishing a series of excitation planes to selectively include a target composition of matter, a means for targeting energy deposition with one or more device or composition of matter absorptive to a predetermined frequency of electromagnetic radiation, a means for localizing a target composition of matter, a means for guiding energy deposition to include all sections of a target composition of matter, or a means for cooling or enhancing cooling capacity of a target substance; or any combination or combinations thereof.

27. An apparatus for depositing energy within an internal part of a member comprising:
a) an antenna;
b) a receptacle that can support a member;
c) a magnet, the magnet being able to produce a magnetic field that can induce nuclear magnetic resonance within at least one composition of matter contained within a member located in the receptacle, whereby a plurality of atomic nuclei in the composition of matter will resonate and can absorb energy from electromagnetic radiation of at least one frequency that can be emitted by the antenna;
d) the antenna, the member, and the magnet being arranged and operated in such a way that the antenna emits electromagnetic radiation, and energy from the electromagnetic radiation is intentionally deposited preferentially within the target substance;
e) whereby for example an operator may cause the deposition of energy within the target substance, which may be a 3-dimensional part contained within the member, which may be a body, and for example a medical technician may cause the heating of diseased or cancerous tissue deep inside a body and thereby precisely treat the tissue or the disease without surgery or other invasive intervention.

28. The apparatus of claim 27, further including a localizing device, such as a digital stereotaxic device, for determining location of the target substance.

29. The apparatus of claim 27, further including a guiding device for assuring that all parts of the target substance can be deposited with the energy, or including a programmable control device for automating the deposition of the energy according to a schedule or prescription such as may be effective for therapy of a medical condition, or including a combination thereof.

30. The apparatus of claim 27, further comprising
a) a set of electromagnets oriented along substantially orthogonal axes, the electromagnets controllably supplementing the fixed magnetic field gradually along the axes to effect a non-homogenous net magnetic field, the net magnetic field being effective to limit to a subset of space the ability of a composition of matter to absorb the emitted electromagnetic radiation;
b) providing an absorptive substance that absorbs a specific frequency of electromagnetic radiation, the absorptive substance being endogenously or interventionally co-located with the target substance;
c) providing in the member or target substance a heat-sensitive substance that is effective only at a temperature above the natural temperature for the member or target substance, and is activated by heat in the region of the target substance, the target substance being preferentially heated by absorption of electromagnetic radiation;
d) a method for dissipating energy in the target substance or surroundings thereof, or both, for example involving:
e) cooling surrounding environment of the member,
f) bathing the member in a cool liquid before, during or after energy deposition,
g) in a case where the target substance is a tissue or bodily region in an organism that has a cardiovascular system, having the organism perform acts that increase the rate or throughput of the cardiovascular system, before, during or after energy deposition, such that more blood may pass through tissue around or in the tissue or bodily region, thereby effecting greater cooling,
h) in a case where the target substance is a tissue or bodily region in an organism that has a cardiovascular system, administering a drug to increase blood flow through tissue around or in the tissue or bodily region, thereby effecting greater cooling,
i) using quick and powerful bursts of electromagnetic radiation to cause a large amount of energy deposition per unit time yet for a short time span, thereby minimizing gross heat transfer and damage to surrounding matter while preserving or enhancing the ability to cause thermal damage to the target substance; or
j) using gradual and small bursts of electromagnetic radiation to cause slow and steady heating, thereby giving the member or target substance time to cool itself according to natural processes,
k) or any combination or combinations thereof; or
l) one or more device requiring power to operate, the device being implanted into the member or target substance, the device being able to be powered by energy absorbed from the electromagnetic radiation;
m) or a combination or combinations thereof.

31. (canceled)

32. A method comprising:
a) providing a first device alike a commercially available magnetic resonance imaging system;
b) providing a member positioned in a standard operating arrangement relative to the first device;
c) providing temperature-mapping protocols for operating the first device, to construct a heat map of the member;
d) imaging the member with the first device;
e) determining a volume of interest containing a target substance within the member, based on the images;
f) heat-mapping the member, including the volume of interest, using the temperature-mapping protocols, to create an initial heat map;
g) prescribing a schedule for heating the target substance, based in part on properties of the target substance, properties of the heat map, and a goal of the prescription;
h) heating the target substance with the first device, operating the first device in a special mode such as is effective to provide the heating;
i) heat-mapping the member using the temperature-mapping protocols, to create a post-heating heat map of the member; and
j) repeating the imaging, heating, and heat-mapping as necessary according to the schedule;
whereby a magnetic resonance imaging system can be operated in three modes, namely imaging, thermometry, and heating, in order to identify, monitor, and intentionally heat a 3-dimensional volume within a member, which can be a body, and for example a mildly skilled operator can precisely and selectively treat cancerous or other diseased tissue deep within a patient's body, without risks, time, expense, need for highly-skilled operators, or scarring associated with conventional surgery or other invasive intervention.

33. The method of claim 32, further comprising:
a) providing a control device to interface with the first device, the control device allowing input from a human operator and interfacing with a control system or console integrated into a magnetic resonance imaging system;
b) providing a database of predetermined protocols to be accessed by the control device, the protocols determining factors for the heating of the target substance, such as durations, pause lengths and amplitudes of pulses of heating, and further factors such as procedures for decomposing the target substance into units that can be individually heated, allowable limits of heating of the target substance and of surrounding substances or classes of substances, and profiles of classes of substances such as tissue types;
c) providing a software means to select from the predetermined protocols or to manually set any factors as may be contained within the protocols; or
d) providing a means to control the first device according to the protocol, to enact all aspects of the protocol;
e) or a combination or combinations thereof;
whereby a highly-skilled operator such as a surgeon may review the images of the member and the target substance and select one of the predetermined protocols or manually create a new one, and a less skilled operator may interact with the control device to carry out the protocol, which automates the imaging, heating and heat-mapping operations, the less-skilled operator thereby providing for instance a treatment for cancer or other non-healthy conditions or states.

34. The method of claim 32, further comprising:
a) providing a tracking device to track motion or conformational changes of the member or the target substance; and
b) using information about the motion or conformational changes to more precisely guide and focus the imaging, thermometry and heating, according to the invention.

35. The method of claim 32, wherein the member or the target substance comprises a body of a human or of a non-human animal, or one or more parts thereof.

36. The method of claim 32, wherein the volume of interest is chosen to demarcate a pathology or cancer within a tissue.

37. The method of claim 32, wherein the target substance comprises at least one biological tissue selected from the group consisting of brain tissue, spinal cord tissue, other neural tissue, cerebrospinal fluid, blood, lymphatic tissue, joints, bones, bone growth plates, bone marrow, arthritic tissue, muscle, veins, arteries, capillaries, heart tissue, lungs, ligaments, tendons, cartilage members, classes of cells, biochemicals, bacteria, viruses, parasites, cancerous tissue, arthritic tissue, transplanted tissue, pathological tissue, lacerated tissue, lesioned tissue, necrotic tissue, epileptic tissue, ischemic tissue, dysmorphic tissue, prolapsed tissue, embryos, hair follicles, skin, dermis, connective tissue, fat, scar tissue, tissue with complex topography such as the deep layers of skin of the entire face, solid waste products, liquid waste products, bodily organs, and organ tissue.

38. The method of claim 32, wherein the member or the target substance comprises at least one item selected from the group consisting of biological samples, animals, plants, food substances, substances containing heat shock proteins, tattoos, semiconductor materials, circuit boards, so-called phantoms generally for calibrating magnetic resonance imaging systems, moldable artistic materials, archaeological samples, prosthetics, plastics, construction materials, cosmetics, liquids, gases, and solids.

39. The method of claim 32, further comprising an analysis of the heat maps and the images in order to determine or verify margins of the target substance, for example when the target substance is a cancerous tumor.

40. The method of claim 32, wherein the temperature-mapping protocols are or are derived from known pulse sequences for measuring heat in a sample using magnetic resonance images, and generating a 3-dimensional map of thermal energy values for each region of the member.

41. The method of claim 32, wherein heating in regions outside of the target substance is monitored and kept within a safe range for human tissue, the safe range being determined according to guidelines for specific absorption ratio for radio-frequency electromagnetic radiation in magnetic resonance imaging, as set by the United States Food and Drug Administration, or equivalent.

42. (canceled)

43. The method of claim 32, wherein the heating of the target substance involves absorption of energy from electromagnetic radiation by a composition of matter within the target substance, the electromagnetic radiation being emitted by the first device.

44-45. (canceled)

46. The method of claim 43, wherein the absorption of energy is concentrated preferentially within the target substance according to an interaction between the electromagnetic radiation and the composition of matter determined by properties of the composition of matter, the composition of matter having been previously deposited preferentially within the target substance by physical or chemical or natural process, whereby an absorptive means in the target substance may be used to guide and focus the heating.

47. The method of claim 32, including a means for selectively causing electromagnetic radiation absorption involving selective imposition of magnetic fields and electromagnetic radiation to converge on the larmour frequency of a target composition of matter such as but not limited to water hydrogen atoms, a means for operating magnetic field gradients and radio-frequency coils of the first device in order to cause selective absorption of radio-frequency energy in a target composition of matter, a means for establishing a series of excitation planes to selectively include a target composition of matter, a means for targeting energy deposition with one or more device or composition of matter absorptive to a predetermined frequency of electromagnetic radiation, a means for localizing a target composition of matter, a means for guiding energy deposition to include all sections of a target composition of matter, a control device to select predetermined or establish a custom protocol for heating a target substance, a tracking device for tracking motion or changes in shape of the target substance, a means for producing heat maps of the member, a means for acquiring images of the member or target substance using the first device according to standard operation of a magnetic resonance imaging system, or a means for cooling or enhancing cooling capacity of a target substance; or any combination or combinations thereof.

48. (canceled)
